(19) United States
(12) Patent Application Publication
(10) Pub. No.: US 2025/0032384 A1
(43) Pub. Date: Jan. 30, 2025

(54) HAIR CARE COMPOSITION

(71) Applicant: Conopco, Inc., d/b/a UNILEVER, Englewood Cliffs, NJ (US)

(72) Inventors: Feng CAO, Shanghai (CN); Guoqiang CHEN, Shanghai (CN); Chengdong JI, Shanghai (CN); Xuezhi TANG, Shanghai (CN); Senpei YANG, Shanghai (CN)

(73) Assignee: Conopco, Inc., d/b/a UNILEVER, Englewood Cliffs, NJ (US)

(21) Appl. No.: 18/714,108

(22) PCT Filed: Nov. 11, 2022
(86) PCT No.: PCT/EP2022/081631

§ 371 Date: May 29, 2024

(30) Foreign Application Priority Data
Nov. 29, 2021 (WO) PCT/CN2021/134083
Jan. 6, 2022 (EP) 22150401.2

(51) Int. Cl. A61K 8/49; A61K 8/19; A61K 8/73; A61Q 5/02

(52) U.S. Cl. CPC A61K 8/4926; A61K 8/19; A61K 8/73; A61Q 5/02

(57) ABSTRACT

A hair care composition is disclosed comprising i) a piroctone compound: ii) a metal oxide or metal salt wherein said metal is selected from magnesium, calcium, barium and strontium; and iii) a cationic guar polymer; wherein the composition comprises at least 2% of said metal oxide or metal salt by weight of the composition.

### FIELD OF THE INVENTION

This invention relates to hair care compositions, more particularly to hair care compositions comprising anti-dandruff agents. The present invention also relates to a method of improving deposition of anti-dandruff agents, especially during topical application to the scalp.

### BACKGROUND OF THE INVENTION

Hair care compositions generally provide cleansing or conditioning benefits or a combination of the two. Such compositions typically comprise one or more cleansing surfactants which generally aid in cleaning the hair and the scalp free of undesirable soil, particles and fatty matter.

Dandruff control is an important aspect of hair cleansing and care. Anti-dandruff benefit has been provided through hair care compositions including shampoos and hair conditioners. Dandruff is an issue that affects many people globally. The condition is manifested by the shedding of clumps of dead skin cells from the scalp. These are white in colour and provide an aesthetically displeasing appearance. A factor that contributes to dandruff is certain members of the Malassezia yeasts. To combat these, anti-dandruff products have included certain anti-dandruff agents which have anti-fungal activity, for example, piroctone compound such as piroctone olamine (Octopirox®). These anti-dandruff agents remove (or at least reduce the level of) the Malassezia from the scalp and provide moderately effective treatment of the dandruff condition. Such a product performs as a hair cleansing shampoo or as a hair conditioner while mitigating the causes of dandruff.

A common problem with piroctone compound is that deposition of the active onto the desired surface during wash process is minimum. The desired surface is typically scalp and/or hair. For example, piroctone compound such as piroctone olamine is usually soluble in surfactants of the cleansing phase comprised in a hair care composition. During the excessive rinsing process, the majority of piroctone is likely to be washed away together with the surfactants. Consequently, there remains a need to improve the deposition of piroctone compounds, onto the surface of scalp and/or hair during washing process.

Cationic guar polymers are often used to enhance the deposition of anti-dandruff agents onto the hair and/or scalp. However, the performance of cationic guar polymer as deposition enhancer is compromised when the anti-dandruff agent is not particulate, and able to dissolve in the surfactants, for example, piroctone compound. The present inventors have now found unexpectedly that the combination of a specific metal oxide or metal salt and cationic guar polymer, can enhance deposition of anti-dandruff agent which is a piroctone compound.

JP 2003146853A (KAWAKEN FINE CHEMICALS CO) is related to the pearly-luster concentrate which is excellent in the washing/cleaning-agent composition and dispersibility which have a pearly luster. It is focused on the stability of pearly luster which is achieved by the combination of N-acyl neutral-amino-acid salt, b-glycol ester, water soluble mineral salt and water

CN 111803392A (BINGO HAIR COSMETIC MFT LTD) discloses a shampoo formulation which can effectively repair damaged hair.

XP055938550 discloses an anti-dandruff shampoo comprising piroctone olamine, calcium chloride and hydroxypropyltrimonium chloride. However, the dosage of calcium chloride is not indicated.

WO 2021224118 A1 (UNILEVER) discloses a composition which can improve the UV stability and deposition efficacy of metal piroctone complex which has a solubility of 2 wt % or less in a 5% aqueous SLES1EO surfactant at 20° C.

### SUMMARY OF THE INVENTION

In a first aspect, the present invention is directed to a hair care composition comprising:
i) a piroctone compound;
ii) a metal oxide or metal salt metal wherein said metal is selected from calcium, barium and strontium; and
iii) a cationic guar polymer;
   wherein the composition comprises at least 2% of said metal oxide or metal salt by weight of the composition.

In a second aspect, present invention is directed to a method of depositing piroctone compound onto scalp comprising the step of applying the hair care composition of the first aspect onto scalp surfaces of an individual followed by rinsing the surfaces with water.

In a third aspect, the present invention is directed to a use of the composition of the first aspect for enhancing deposition of piroctone compound onto scalp.

All other aspects of the present invention will more readily become apparent upon considering the detailed description and examples which follow.

### DETAILED DESCRIPTION OF THE INVENTION

### Hair Care Composition

“Hair care composition”, as used herein, is meant to include a composition for topical application to hair and/or scalp of mammals, especially humans. Such a composition may be generally classified as leave-on or rinse off, and includes any product applied to a human body for also improving appearance, cleansing, odor control or general aesthetics. The composition of the present invention can be in the form of a liquid, lotion, cream, foam, scrub, gel, or bar. Non-limiting examples of such compositions include leave-on hair lotions, creams, and rinse-off shampoos, conditioners, shower gels, or toilet bar. The composition of the present invention is preferably a rinse-off composition, especially preferred being a shampoo or a conditioner and most preferably a shampoo.

### Molecular Weight

“Molecular weight”, as used herein, refers to the weight average molecular mass of a given polymer. The weight average molecular weight (WAVG MW) of a given polymer is determined by SEC (Size Exclusion Chromatography) analysis using absolute calibration (universal calibration). Polysaccharide standards pulluan and dextran were used for calibration.

### Miscellaneous

Except in the examples, or where otherwise explicitly indicated, all numbers in this description indicating amounts of material or conditions of reaction, physical properties of materials and/or use may optionally be understood as modified by the word “about”.

All amounts are by weight of the final hair care composition, unless otherwise specified. It should be noted that in specifying any ranges of values, any particular upper value can be associated with any particular lower value.

For the avoidance of doubt, the word “comprising” is intended to mean “including” but not necessarily “consisting of” or “composed of”. In other words, the listed steps or options need not be exhaustive.

The disclosure of the invention as found herein is to be considered to cover all embodiments as found in the claims as being multiply dependent upon each other irrespective of the fact that claims may be found without multiple dependency or redundancy.

Where a feature is disclosed with respect to a particular aspect of the invention (for example a composition of the invention), such disclosure is also to be considered to apply to any other aspect of the invention (for example a method of the invention) mutatis mutandis.

### Piroctone Compound

The piroctone compound for use in the present invention include piroctone acid, primary, secondary and tertiary olamine salts of piroctone acid (such as the diethanolamine and triethanolamine salts), and mixtures thereof, preferably piroctone acid, primary olamine salt of piroctone acid (i.e. piroctone olamine, also known as Octopirox®) and mixtures thereof. It is particularly preferred that the piroctone compound is piroctone olamine.

Piroctone olamine is an olamine salt of the hydroxamic acid derivative piroctone. It is commonly known as piroctone ethanolamine with the trade name Octopirox®.

The piroctone olamine according to the present invention is a 1:1 compound of 1-hydroxy-4-methyl-6-(2,4,4-trimethylpentyl)-2 (1H)-pyridinone with 2-aminoethanol and is also designated 1-hydroxy-4-methyl-6-(2,4,4-trimethylpentyl)-2 (1H) pyridinone monoethanolamine salt. The CAS number is 68890-66-4 and the compound has the general formula (I) as below:

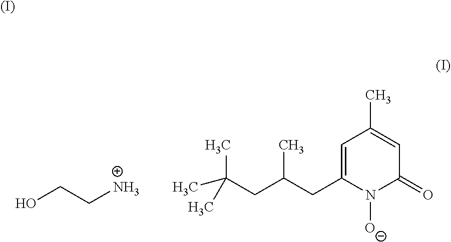

The piroctone compound, particularly preferred the piroctone olamine, is preferably present from 0.01 to 10%, more preferably from 0.05 to 5 w %, furthermore preferably from 0.1 to 2%, most preferably from 0.2 to 1.5% by weight of the composition.

### Cationic Guar Polymer

The composition of the present invention comprises a cationic guar polymer.

Preferably, the composition comprises cationic guar polymer having a molecular weight of from 0.3 to 2.5 million Dalton and a cationic degree of substitution of from 0.05 to 0.4.

It is preferred that the cationic guar polymer has a molecular weight of from 1.0 to 2.3 million Dalton, more preferably from 1.0 to 1.5 million Dalton.

It is preferred that the cationic guar polymer has a cationic degree of substitution of from 0.1 to 0.3, more preferably from 0.16 to 0.2.

It is most preferred that cationic guar polymer has a molecular weight of from 1.0 million to 1.5 million Dalton and a cationic degree of substitution (DS) of from 0.16 to 0.20. This DS value corresponds to a charge density of from 0.85 to 1.05.

Cationic guar polymer is preferably guar hydroxypropyltrimonium chloride. Guar polymer predominantly contains galactomannan polymer chains. This polymer is available at various molecular weights and degree of cationic substitutions depending on how much the guar has been hydrolysed and cationised.

Generally for cationic polysaccharide polymers, the hydroxyl groups of the non-modified monomeric sugar ring units are the sites for cationic substitution. Degree of substitution (DS) is typically in the range from 0 to 3 due to the fact that the monomeric sugar unit of most polysaccharide has in average three hydroxyl groups available for substitution. In addition to the DS, the cationic charge on polymers can also be quantified as cationic charge density. DS has previously been determined by different methods. For example, the cationic charge density of the polymer has in some cases been calculated based on a percent nitrogen content determined via the Kjeldahl method as described in US Pharmacopoeia under chemical tests for nitrogen determination and is expressed in milliequivalents (meq) per gram. The cationic charge density of the polymer in the present invention is, however, calculated from the degree of substitution, which is measured by 1H NMR in a solvent of deuterium oxide (D2O) and deuterium chloride (DCI) mixture.

In many cases the DS obtained from 1H NMR measurement may not be suitable to be compared with that obtained from Kjeldahl method, due to the fact that the two methods are influenced by different factors.

In the wide spectrum of molecular weights of guars available, the cationic guar for use in the present invention has a molecule weight which is considered a ‘medium’ molecular weight. In the wide range of charge densities, the above range for use in the present invention is considered a ‘medium’ range.

The cationic guar polymer is preferably present in an amount of from 0.001 to 2%, more preferably from 0.01 to 1%, furthermore preferably 0.02 to 0.8%, most preferably from 0.05 to 0.5% by weight of the composition.

### Metal Oxide or Metal Salt

The composition of the present invention comprises a metal oxide or metal salt wherein the metal is selected from calcium, barium and strontium.

When the composition comprises a metal oxide, it is preferably selected form calcium oxide, strontium oxide and barium oxide.

When the composition comprises a salt of calcium, it is preferably selected from calcium PCA, calcium pantothenate, calcium chloride, calcium gluconate, calcium carbonate and calcium silicate.

When the composition comprises a salt of strontium, it is preferably selected from strontium chloride, strontium acetate, strontium nitrate, strontium hydroxide and strontium thioglycolate.

When the composition comprises a salt of barium, it is preferably selected from barium sulfate, barium sulfide, barium carbonate, barium chloride and barium silicate.

The metal oxide or metal salt of the present invention is present in an amount of at least 2%, preferably from 2 to 10%, more preferably from 2 to 5%, furthermore preferably from 2 to 3% by weight of the composition.

Without wishing to be bound by theory, the present inventors believe that the specific metal oxide or metal salt can form complex with the piroctone compound, therefore the deposition of piroctone compound can be improved with the presence of the cationic guar polymer.

It is preferred that the weight ratio of metal oxide or metal salt to the piroctone compound is from 1:1 to 20:1, preferably from 2:1 to 17:1, more preferably from 4:1 to 10:1.

### Cleansing Surfactant

The composition of this invention preferably comprises a cleansing surfactant comprising an anionic surfactant.

Non-limiting examples of suitable anionic surfactants are alkyl sulphates, alkyl ether sulphates, alkaryl sulphonates, alkanoyl isethionates, alkyl succinates, alkyl sulphosuccinates, alkyl ether sulphosuccinates, N-alkyl sarcosinates, alkyl phosphates, alkyl ether phosphates, and alkyl ether carboxylic acids and salts thereof, especially their sodium, magnesium, ammonium and mono-, di- and triethanolamine salts. The alkyl and acyl groups generally contain from 8 to 18, preferably from 10 to 16 carbon atoms and may be unsaturated. The alkyl ether sulphates, alkyl ether sulphosuccinates, alkyl ether phosphates and alkyl ether carboxylic acids and salts thereof may contain from 1 to 20 ethylene oxide or propylene oxide units per molecule. Typical anionic surfactants for use in compositions of the invention include, but not limited to, sodium oleyl succinate, ammonium lauryl sulphosuccinate, sodium lauryl sulphate, sodium lauryl ether sulphate, sodium lauryl ether sulphosuccinate, ammonium lauryl sulphate, sodium dodecylbenzene sulphonate, triethanolamine dodecylbenzene sulphonate, sodium cocoyl isethionate, sodium lauryl isethionate, lauryl ether carboxylic acid, sodium N-lauryl sarcosinate or mixtures thereof.

Preferred anionic surfactants are the alkyl sulphates and alkyl ether sulphates. Preferred alkyl sulphates are C8-18 alky sulphates, more preferably C12-18 alkyl sulphates, preferably in the form of a salt with a solubilising cation such as sodium, potassium, ammonium or substituted ammonium. Examples are sodium lauryl sulphate (SLS) or sodium dodecyl sulphate (SDS). It is particularly preferred that the anionic surfactant is alkyl ether sulphate. Preferred alkyl ether sulphates are those having the formula: RO(CH2CH2O)nSO-3M; wherein R is an alkyl or alkenyl having from 8 to 18 (preferably 12 to 18) carbon atoms; n is a number having an average value of greater than at least 0.5, preferably between 1 and 3, more preferably between 1 and 2; and M is a solubilising cation such as sodium, potassium, ammonium or substituted ammonium. An example is sodium lauryl ether sulphate (SLES). Preferred alkyl ether sulphate is sodium lauryl ether sulphate having an average degree of ethoxylation of from 0.5 to 3, preferably from 1 to 3, more preferably from 1 to 2.

It is preferred that the anionic surfactant is an ethoxylated alkyl sulphate a degree of ethoxylation of less than 3, preferably an ethoxylated alkyl sulphate a degree of ethoxylation of less than 2, more preferably sodium laureth sulphate (1EO).

The anionic surfactants are typically present in hair care composition of the present invention at a level of from 0.5 to 20%, more preferably from 2 to 16% and most preferably from 3 to 16%, by weight of the hair care composition.

Suitable amphoteric surfactant examples include alkyl amine oxides, alkyl betaines, alkyl amidopropyl betaines, alkyl sulphobetaines (sultaines), alkyl amphoacetates, alkyl amphopropionates, alkyl amidopropyl hydroxysultaines, wherein the alkyl group has from 8 to 19 carbon atoms.

Preferably, the amphoteric surfactant is a betaine surfactant. It is preferred that the betaine surfactant is one of alkyl amidopropyl betaines. Cocamidopropyl betaine (CAPB) is particularly preferred.

In a preferred embodiment, the composition comprises from 0.1 to 10 wt. %, preferably from 0.5 to 8 wt. %, more preferably from 1 to 5 wt. % of an amphoteric surfactant by weight of the total composition.

The ratio of ethoxylated anionic surfactant to amphoteric surfactant in which the ratio of anionic surfactant to amphoteric surfactant is less than 10:1, preferably from 2:1 to 10:1, more preferably from 3:1 to 9:1.

The total amount of cleansing surfactant in a shampoo composition for use in the invention is generally from 3 to 35 wt %, preferably from 5 to 25 wt %, most preferably from 7 to 16 wt % of the total composition.

### Hair Care Composition

The hair care composition in accordance with the present invention comprises an aqueous cosmetically acceptable carrier.

In a preferred embodiment, the hair care composition is a shampoo, particularly a rinse off shampoo. Shampoo compositions for use in the invention are generally aqueous, i.e. they have water or an aqueous solution or a lyotropic liquid crystalline phase as their major component. Suitably, the shampoo composition will comprise from 50 to 98%, preferably from 60 to 92% water by weight based on the total weight of the composition. Such compositions are referred to as having an aqueous base.

It is preferable that the hair care composition also comprises other cationic polymers. Suitable cationic polymers may be homopolymers or be formed from two or more types of monomers. The molecular weight of the polymer will generally be between 5,000 and 10,000,000 g/mol, typically at least 10,000 g/mol and preferably from 100,000 to 2,000,000 g/mol. The polymers will have cationic nitrogen containing groups such as quaternary ammonium or protonated amino groups, or a mixture thereof. The cationic nitrogen containing group will generally be present as a substituent on a fraction of the total monomer units of the cationic polymer. Thus when the polymer is not a homopolymer it can contain spacer non-cationic monomer units. The ratio of the cationic to non-cationic monomer units is selected to give a polymer having a cationic charge density in the required range.

Suitable cationic polymers include, for example, copolymers of vinyl monomers having cationic amine or quaternary ammonium functionalities with water soluble spacer monomers such as (meth)acrylamide, alkyl and dialkyl (meth)acrylamides, alkyl (meth)acrylate, vinyl caprolactone and vinyl pyrrolidine. The alkyl and dialkyl substituted monomers preferably have C1-C7 alkyl groups, more preferably C1-C3 alkyl groups. Other suitable spacers include vinyl esters, vinyl alcohol, maleic anhydride, propylene glycol and ethylene glycol.

Preferably, the cationic polymer is a cationic polysaccharide polymer such as cationic guar, cationic starch, and cationic cellulose. Suitably, such cationic polysaccharide polymers have a molecular weight of from 100,000 g/mol to 2,300,000 g/mol, more preferably from 150,000 g/mol to 2,000,000 g/mol. Such cationic polysaccharide polymers preferably have a cationic charge density from 0.1 to 4 meq/g.

Cationic polysaccharide polymers suitable for use in compositions of this invention include those represented by the general formula:

A-O—[R1-N+(R2)(R3)(R4)X—]

wherein: A is an anhydroglucose residual group, such as a starch or cellulose anhydroglucose residual. R1 is an alkylene, oxyalkylene, polyoxyalkylene, or hydroxyalkylene group, or combination thereof. R2, R3 and R4 independently represent alkyl, aryl, alkylaryl, arylalkyl, alkoxyalkyl, or alkoxyaryl groups, each group containing up to about 18 carbon atoms. The total number of carbon atoms for each cationic moiety (i.e., the sum of carbon atoms in R2, R3 and R4) is preferably about 20 or less, and X is an anionic counterion.

Cationic cellulose is available from Amerchol Corp. (Edison, NJ, USA) in their Polymer JR (trade mark) and LR (trade mark) series of polymers, as salts of hydroxyethyl cellulose reacted with trimethyl ammonium substituted epoxide, referred to in the industry (CTFA) as Polyquaternium 10. Another type of cationic cellulose includes the polymeric quaternary ammonium salts of hydroxyethyl cellulose reacted with lauryl dimethyl ammonium-substituted epoxide, referred to in the industry (CTFA) as Polyquaternium 24. These materials are available from Amerchol Corp. (Edison, NJ, USA) under the tradename Polymer LM-200.

Other suitable cationic polysaccharide polymers include quaternary nitrogen-containing cellulose ethers (e.g. as described in U.S. Pat. No. 3,962,418) and copolymers of etherified cellulose and starch (e.g. as described in U.S. Pat. No. 3,958,581).

When used, the cationic polymer will generally be present in the hair care composition of the present invention in an amount of from 0.001 to 1% by weight of the hair care composition, more preferably from 0.01 to 0.5%, and most preferably from 0.03 to 0.3%, based on total weight of the hair care composition and including all ranges subsumed therein.

The hair care composition may additionally comprise a conditioning agent to provide conditioning benefit. Preferably, the hair care composition comprises discrete dispersed droplets of a water-insoluble conditioning agent, which has a mean droplet diameter (D3,2) of less than 15 microns, preferably less than 10 microns, more preferably less than 5 microns, most preferably less than 3 microns. The mean droplet diameter (D3,2) of a water-insoluble conditioning agent may be measured by means of a laser light scattering technique, for example using a 2600D ParticleSizer from Malvern Instruments.

The water-insoluble conditioning agent may include non-silicone conditioning agent comprising non-silicone oily or fatty materials such as hydrocarbon oils, fatty esters and mixtures thereof. Preferably, the water-insoluble conditioning agent is emulsified silicone oil.

Suitable silicones include polydiorganosiloxanes, in particular polydimethylsiloxanes which have the CTFA designation dimethicone. Also suitable for use in compositions of this invention (particularly shampoos and conditioners) are polydimethyl siloxanes having hydroxyl end groups, which have the CTFA designation dimethiconol. Also suitable for use in compositions of this invention are silicone gums having a slight degree of cross-linking, as are described for example in WO 96/31188. Preferably, the silicone oil comprises dimethicone, dimethiconol or a mixture thereof.

Suitable emulsified silicones for use in the hair care compositions of this invention are available as pre-formed silicone emulsions from suppliers of silicones such as Dow Corning and GE silicones. The use of such pre-formed silicone emulsion is preferred for ease of processing and control of silicone particle size. Such pre-formed silicone emulsions will typically additionally comprise a suitable emulsifier, and may be prepared by a chemical emulsification process such as emulsion polymerisation, or by mechanical emulsification using a high shear mixer. Examples of suitable pre-formed silicone emulsions include DC1785, DC1788, DC7128, all available from Dow Corning. These are emulsions of dimethiconol/dimethicone.

Another class of silicones which may be used are functionalized silicones such as amino functional silicones, meaning a silicone containing at least one primary, secondary or tertiary amine group, or a quaternary ammonium group. Examples of suitable amino functional silicones include polysiloxanes having the CTFA designation “amodimethicone.”

The water-insoluble conditioning agent is generally present in hair care composition of this invention in an amount from 0.05 to 15%, preferably from 0.1 to 10%, more preferably from 0.5 to 8%, most preferably from 1 to 5%, based on total weight of the hair care composition and including all ranges subsumed therein.

The water-insoluble conditioning agent may include non-silicone conditioning agent comprising non-silicone oily or fatty materials such as hydrocarbon oils, fatty esters and mixtures thereof. Preferably the composition of the invention further comprises a suspending agent. Suitable suspending agents are selected from polyacrylic acids, cross-linked polymers of acrylic acid, copolymers of acrylic acid with a hydrophobic monomer, copolymers of carboxylic acid-containing monomers and acrylic esters, cross-linked copolymers of acrylic acid and acrylate esters, heteropolysaccharide gums and crystalline long chain acyl derivatives. The long chain acyl derivative is desirably selected from ethylene glycol stearate, alkanolamides of fatty acids having from 16 to 22 carbon atoms and mixtures thereof. Ethylene glycol distearate and polyethylene glycol 3 distearate are preferred long chain acyl derivatives, since these impart pearlescence to the composition. Polyacrylic acid is available commercially as Carbopol 420, Carbopol 488 or Carbopol 493. Polymers of acrylic acid cross-linked with a polyfunctional agent may also be used; they are available commercially as Carbopol 910, Carbopol 934, Carbopol 941 and Carbopol 980. An example of a suitable copolymer of a carboxylic acid containing monomer and acrylic acid esters is Carbopol 1342. All Carbopol (trademark) materials are available from Goodrich.

Suitable cross-linked polymers of acrylic acid and acrylate esters are Pemulen TR1 or Pemulen TR2. A suitable heteropolysaccharide gum is xanthan gum, for example that available as Kelzan mu.

Mixtures of any of the above suspending agents may be used. Preferred is a mixture of cross-linked polymer of acrylic acid and crystalline long chain acyl derivative.

The suspending agent is generally present in hair care composition of this invention in an amount of from 0.1 to 10%, more preferably from 0.2 to 6%, and most preferably from 0.3 to 4%, based on total weight of the hair care composition and including all ranges subsumed therein.

Preservatives may also be incorporated into the hair care composition of this invention to protect against the growth of potentially harmful microorganisms. Suitable traditional preservatives include alkyl esters of parahydroxybenzoic acid, hydantoin derivatives, propionate salts, and a variety of quaternary ammonium compounds. Illustrative yet non-limiting examples of the types of preservatives that may be used in this invention include, for examples, phenoxyethanol, sodium salicylate, methyl paraben, butyl paraben, propyl paraben, diazolidinyl urea, sodium dehydroacetate, benzyl alcohol, sodium benzoate, iodopropynyl butylcarbamate, caprylyl glycol, disodium EDTA or mixtures thereof. In an especially preferred embodiment, the preservative is sodium benzoate, phenoxyethanol, sodium salicylate or a mixture thereof.

Preservatives are preferably employed in amounts ranging from 0.01 to 2% by weight of the hair care composition.

The hair care composition of the present invention may contain other ingredients which are common in the art to enhance physical properties and performances. Suitable ingredients include but are not limited to fragrance, dyes and pigments, pH adjusting agents, pearlescers or opacifiers, viscosity modifiers, thickeners, and natural hair nutrients such as botanicals, fruit extracts, sugar derivatives and amino acids.

### Method and Use

The present invention provides for a method of depositing piroctone compound onto scalp comprising the step of applying the hair care composition of the first aspect onto scalp surfaces of an individual followed by rinsing the surfaces with water. The method is non-therapeutic in nature. Preferably it is for cosmetic purpose. It is preferred that the piroctone compound is piroctone olamine.

The present invention also provides for use of the composition of the first aspect for enhancing deposition of piroctone compound onto scalp. The use is non-therapeutic in nature. Preferably it is for cosmetic purpose. It is preferred that the piroctone compound is piroctone olamine.

The present invention also provides for a composition of the first aspect for use in preventing or alleviating the symptoms of dandruff on the scalp or hair.

### Mode of Use

The compositions of the invention are primarily intended for topical application to scalp and/or at least a portion of the hair of an individual, either in rinse-off or leave-on compositions, preferably in rinse-off compositions like shampoos.

When the composition is a shampoo, it is topically applied to the hair and then massaged into the hair and scalp. Then it is rinsed with water prior to drying the hair.

The invention will be further illustrated by the following, non-limiting Examples, in which all percentages quoted are by weight based on total weight unless otherwise stated.

The examples are intended to illustrate the invention and are not intended to limit the invention to those examples per se.

### EXAMPLES

### Example 1

This example demonstrated the combination of metal oxide or metal salt and cationic guar polymer within the scope of the present invention can improve the deposition of antidandruff agent which is a piroctone compound. The compositions according to the formulations detailed in Table 1 are prepared. All ingredients are expressed by weight percent of the total formulation, and as level of active ingredient.

TABLE 1

| | Samples | | | | | |
|---|---|---|---|---|---|---|
| Ingredient | A | B | C | D | E | 1 |
| Water | To | To | To | To | To | To |
| | 100 | 100 | 100 | 100 | 100 | 100 |
| Sodium lauryl sulphate | 13.00 | 13.00 | 13.00 | 13.00 | 13.00 | 13.00 |
| Cocoamidopropyl betaine | 1.50 | 1.50 | 1.50 | 1.50 | 1.50 | 1.50 |
| Carbomer | 0.35 | 0.35 | 0.35 | 0.35 | 0.35 | 0.35 |
| Guar | — | 0.20 | — | — | 0.20 | 0.20 |
| hydroxypropyltrimonium | | | | | | |
| chloride^{a} | | | | | | |
| Piroctone olamine | 0.50 | 0.50 | 0.50 | 0.50 | 0.50 | 0.50 |
| Calcium chloride | — | — | 1.00 | 2.00 | 1.00 | 2.00 |

^{a}Commercial guar hydroxypropyltrimonium chloride has a DS of 0.16 to 0.20 and a weight average molecular weight from 1.0 to 1.5 million g/mol under the trade name BB-18 from Lamberti.

### Methods

About 0.2 grams of the test sample was taken on artificial skin (VITRO-SKIN from IMS testing group). This was diluted with 1.8 mL water and rubbed with a plastic rod for 30 seconds. The artificial skin surface was then rinsed twice with water, first time with 4 mL water for 30 second and then again with 4 mL water for 30 seconds. The deposition of piroctone olamine on the skin (10.75 cm² per plate) was measured using HPLC method.

### Results

The average deposition (of five such experiments) is summarized in Table 2 (error represents standard deviation for duplicate measurements).

TABLE 2

| Samples | A | B | C | D | E | 1 |
|---|---|---|---|---|---|---|
| Piroctone | 12.98 ± | 12.00 ± | 11.36 ± | 12.6 ± | 13.76 ± | 15.62 ± |
| olamine | 0.30 | 0.73 | 0.64 | 1.00 | 1.65 | 0.27 |
| deposition | | | | | | |
| (μg/plate) | | | | | | |

Samples 1 comprising combination of metal salt and cationic guar polymer within the scope of the present invention showed significantly better deposition of piroctone olamine as compared to samples A-E which are outside the scope of the present invention.

### Example 2

This example demonstrated the combination of cationic guar polymer and metal oxide or metal salt outside the scope of the present invention cannot deliver the enhanced deposition of antidandruff agent which is piroctone compound. The compositions according to the formulations detailed in Table 3 are prepared. All ingredients are expressed by weight percent of the total formulation, and as level of active ingredient.

TABLE 3

| | Samples | | | |
|---|---|---|---|---|
| Ingredient | F | G | H | I |
| Water | To 100 | To 100 | To 100 | To 100 |
| Sodium lauryl sulphate | 13.00 | 13.00 | 13.00 | 13.00 |
| Cocoamidopropyl betaine | 1.50 | 1.50 | 1.50 | 1.50 |
| Carbomer | 0.35 | 0.35 | 0.35 | 0.35 |
| Guar hydroxypropyltrimonium | — | 0.20 | 0.20 | 0.20 |
| chloride^{a} | | | | |
| Piroctone olamine | 0.50 | 0.50 | 0.50 | 0.50 |
| Zinc oxide | — | — | 2.00 | — |
| Zin chloride | — | — | — | 2.00 |

^{a}Commercial guar hydroxypropyltrimonium chloride has a DS of 0.16 to 0.20 and a weight average molecular weight from 1.0 to 1.5 million g/mol under the trade name BB-18 from Lamberti.

### Methods

Same method was used as disclosed in Example 1.

### Results

The average deposition (of five such experiments) is summarized in Table 4 (error represents standard deviation for duplicate measurements).

TABLE 4

| Samples | F | G | H | I |
|---|---|---|---|---|
| Piroctone olamine | 13.98 ± | 14.44 ± | 8.47 ± | 9.21 ± |
| deposition (μg/plate) | 0.44 | 1.14 | 0.80 | 2.80 |

The results above indicates that samples comprising cationic guar polymer and the metal oxide or metal salts outside the scope of the present invention cannot improve the deposition of the piroctone compound.

### Example 3

This example demonstrated the combination of metal oxide or metal salt and cationic guar polymer within the scope of the present invention can improve the deposition of antidandruff agent which is a piroctone compound. The compositions according to the formulations detailed in Table 5 are prepared. All ingredients are expressed by weight percent of the total formulation, and as level of active ingredient.

TABLE 5

Samples

| Ingredient | J | 2 | 3 |
|---|---|---|---|
| Water | To 100 | To 100 | To 100 |
| Sodium lauryl sulphate | 13.00 | 13.00 | 13.00 |
| Cocoamidopropyl betaine | 1.50 | 1.50 | 1.50 |
| Carbomer | 0.35 | 0.35 | 0.35 |
| Guar hydroxypropyltrimonium | 0.20 | 0.20 | 0.20 |
| chloride^{a} | | | |
| Piroctone olamine | 0.50 | 0.50 | 0.50 |
| Barium chloride | — | 2.00 | — |
| Strontium acetate | — | — | 2.00 |

^{a}Commercial guar hydroxypropyltrimonium chloride has a DS of 0.16 to 0.20 and a weight average molecular weight from 1.0 to 1.5 million g/mol under the trade name BB-18 from Lamberti.

### Methods

Same method was used as disclosed in Example 1.

### Results

The average deposition (of five such experiments) is summarized in Table 6 (error represents standard deviation for duplicate measurements).

TABLE 6

| Samples | J | 2 | 3 |
|---|---|---|---|
| Piroctone olamine deposition | 12.00 ± 0.24 | 18.85 ± 1.6 | 26.41 ± 0.64 |
| (μg/plate) | | | |

Samples 2-3 comprising combination of metal salt and cationic guar polymer within the scope of the present invention showed significantly better deposition of piroctone olamine as compared to samples J which is outside the scope of the present invention.

1. A hair care composition comprising:
i) a piroctone compound;
ii) a metal oxide or a metal salt wherein the metal is selected from calcium, barium and strontium; and
iii) a cationic guar polymer;
wherein the composition comprises at least 2% of the metal oxide or the metal salt by weight of the composition.

2. The composition as claimed in claim 1, wherein the metal salt is a salt of calcium selected from calcium PCA, calcium pantothenate, calcium chloride, calcium gluconate, calcium carbonate and calcium silicate.

3. The composition as claimed in claim 1, wherein the metal salt is a salt of strontium selected from strontium chloride, strontium acetate, strontium nitrate, strontium hydroxide and strontium thioglycolate.

4. The composition as claimed in claim 1, wherein the metal salt is a salt of barium selected from barium sulfate, barium sulfide, barium carbonate, barium chloride and barium silicate.

5. The composition as claimed in claim 1, wherein the metal oxide is selected from magnesium oxide, calcium oxide, strontium oxide and barium oxide.

6. The composition as claimed in claim 1, wherein the piroctone compound is piroctone olamine.

7. The composition as claimed in claim 1, wherein the cationic guar polymer has a molecular weight of from 0.3 to 2.5 million Dalton and a cationic degree of substitution of from 0.05 to 0.4.

8. The composition as claimed in claim 1, wherein the cationic guar polymer is guar hydroxypropyl trimonium chloride.

9. The composition as claimed in claim 1, wherein the composition comprises the cationic guar polymer in an amount of from 0.01 to 2%.

10. The composition as claimed in claim 1, wherein the composition comprises the piroctone compound in an amount of from 0.01 to 10%.

11. The composition as claimed in claim 1, wherein the composition comprises the metal oxide or the metal salt in an amount from 2 to 10%, by weight of the composition.

12. The composition as claimed in claim 1, further comprising an anionic surfactant.

13. The composition as claimed in wherein said composition is a shampoo.

14. A method of depositing piroctone compound onto a scalp comprising:
applying the composition as claimed claim 1 onto the scalp of an individual; and
rinsing the scalp with water.

15. (canceled)

16. The composition as claimed in claim 7, wherein the cationic guar polymer has a molecular weight of from 1.0 to 2.3 million Dalton and a cationic degree of substitution of from 0.1 to 0.3.

17. The composition as claimed in claim 1, wherein a weight ratio of the metal oxide or the metal salt to the piroctone compound is from 4:1 to 10:1.

18. The composition as claimed in claim 12, wherein the anionic surfactant, is sodium lauryl ether sulphate.

19. The composition as claimed in claim 12 further comprising at least one amphoteric surfactant selected from alkyl amine oxides, alkyl betaines, alkyl amidopropyl betaines, alkyl sulphobetaines (sultaines), alkyl amphoacetates, alkyl amphopropionates, alkyl amidopropyl hydroxysultaines, wherein the alkyl group has from 8 to 19 carbon atoms.

20. The composition as claimed in claim 19, wherein a weight ration of anionic surfactant to amphoteric surfactant is from 3:1 to 9:1.

21. The composition as claimed in claim 19, wherein a total amount of anionic and amphoteric surfactant is from 7 to 16% by weight of the composition.
